(19) United States
(12) Patent Application Publication
(10) Pub. No.: US 2009/0234454 A1
(43) Pub. Date: Sep. 17, 2009

(54) Spinal Implant With Structural Support And Bone Interface Surfaces Formed from UHMWPE

(75) Inventor: Tzony Siegal, Moshav Shoeva (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: NONLINEAR TECHNOLOGIES LTD., Ariel (IL)

(21) Appl. No.: 12/402,621

(22) Filed: Mar. 12, 2009

Related U.S. Application Data
(60) Provisional application No. 61/035,763, filed on Mar. 12, 2008.

(51) Int. Cl. A61F 2/44

(52) U.S. Cl. 623/17.11

(57) ABSTRACT

A spinal implant and corresponding method for deploying a spinal implant in which both the structural support and the bone interface surfaces are formed from ultra-high-molecular-weight polyethylene (UHMWPE). Examples disclosed include intervertebral disc replacements and interspinous process spacers.

This applications claims the benefit of provisional application No. 61/035,763 filed Mar. 12, 2008.

### FIELD AND BACKGROUND OF THE INVENTION

The present invention relates to spinal implants and, in particular, it concerns a spinal implant in which both the structural support and the bone interface surfaces formed from ultra-high-molecular-weight polyethylene (UHMWPE).

Ultra-high-molecular-weight polyethylene (UHMWPE) has been used in total hip replacement medical implants for several decades, primarily as a bearing surface liner within a metal prosthetic acetabular cup. An example of such an application may be found in U.S. Pat. No. RE28895. UHMWPE is a preferred choice for such bearing surfaces due to its very high resistance to wear, and its self-lubricating low-friction properties.

Despite its widespread clinical use in the context of bearing surfaces for total joint replacements, and certain other use for internal components of other implants, UHMWPE has not been adopted as a bulk material for constructing other implants due to a number of shortcomings. One problem, discussed at length in a paper “Physicochemical and mechanical properties of UHMWPE 45 years' experience” by L. Costa et al. (Interact Surg (2007) 2: 169-173), is that of debris formed by mechanical wear under conditions of cyclic loading. According to Costa et al., “Polyethylene debris, formed during in vivo implantation, initiate an inflammatory reaction, the formation of a loosening membrane, and a secondary osteolysis; usually junctional tissue is related with the number and size of UHMWPE debris . . . the debris is not just simple UHMWPE particles. If the implanted component had been sterilised with gamma radiation in air, it can be variable degraded and oxidised and the resulting debris cannot be referred to as UHMWPE debris, but rather as an oxidised, lower molecular mass polyethylene debris. Furthermore, debris is biologically active, independently of the sterilisation process, since it is made of particles whose surface can interact with the human tissues . . . . Whereas dramatic failures due to anomalous wear of heavily oxidised polyethylene have become quite uncommon nowadays for the new prosthetic components in UHMWPE, abrasion and production of abraded particles remain a problem in young patients whose life expectancy and quality of life are very high. The abrasion is the particle loss due to friction caused by the reciprocal movement of the loaded articular surfaces, for equal mechanical stress, material and interface. It is a function of time.”

Another problem associated with use of UHMWPE as a primary structural component of an implant is that of “flow” or “creep”, as discussed in U.S. Pat. No. 5,645,594. Since the glass transition temperature of UHMWPE is below body temperature, slight changes in dimensions or surface geometry of the implant would be expected to occur over time. For this reason, conventional teaching requires UHMWPE to be used as an internal component of an implant where all bone contact surfaces are provided by components formed largely or entirely from other materials.

In the field of spinal surgery, many types of implants are formed primarily from hard metallic components. These metallic components suffer from problems of subsidence, where they tend to sink into the adjacent bone structures under applied loading, and cause relatively rapid wear of the adjacent bones as a result of wear.

UHMWPE has also been introduced as a bearing material in a total disc replacement product, as exemplified by the product marketed under the name PRODISC®, commercially available from Synthes Inc. (US). Here too, upper and lower endplates of the implant are made from metal, thereby defining boundaries of the UHMWPE against creep and limiting exposure of the surrounding tissue to debris.

It would therefore be highly advantageous to provide spinal implants which would reduce problems of subsidence and bone wear.

### SUMMARY OF THE INVENTION

The present invention is a spinal implant in which both the structural support and the bone interface surfaces are formed from UHMWPE.

For the purpose of the description and claims, the term ultra-high-molecular-weight polyethylene or UHMWPE is used to refer to long chain polyethylene with molecular weight in excess of 1 million. The term may refer to simple UHMWPE or UHMWPE which has undergone additional treatment to achieve cross-linking of molecular chains. Unless otherwise qualified, the term as used herein also encompasses admixtures of various other minority materials with UHMWPE in order to modify or improve certain properties or features of the material, so long as the UHMWPE remains the predominant factor in determining the overall properties of the material. Preferably, the invention is implemented using material which is essentially UHMWPE (i.e., at least 96%), and most preferably using pure UHMWPE.

According to the teachings of the present invention there is provided, a spinal implant for implantation in a human spine adjacent to at least one bone, the spinal implant comprising an implant structure including at least one bone contact surface, wherein a majority of the implant structure by volume is formed from UHMWPE, and wherein the UHMWPE provides the at least one bone contact surface.

There is also provided according to the teachings of the present invention, a method for deploying a spinal implant comprising the steps of: (a) providing an implant structure including at least one bone contact surface, wherein a majority of the implant structure by volume is formed from UHMWPE, and wherein the UHMWPE provides the at least one bone contact surface; and (b) deploying the implant structure with the at least one bone contact surface in contact with a bone of a human spine.

According to a further feature of the present invention, the implant structure is shaped for deployment as an inter spinous process spacer such that both an inferior and a superior spinous process come in contact with the UHMWPE.

According to a further feature of the present invention, the implant structure is shaped for deployment as an intervertebral disc replacement such that both an inferior and a superior vertebral body endplate come in contact with the UHMWPE.

According to a further feature of the present invention, the implant structure is shaped for deployment at least partially within a vertebral body.

According to a further feature of the present invention, the implant structure includes a plurality of segments interconnected by at least one integral hinge, the segments and the integral hinges being formed from the UHMWPE.

According to a further feature of the present invention, the implant structure includes a locking arrangement including at least one resilient locking element resiliently biased to a locking position such that, when the implant structure is deflected by flexing at the at least one integral hinge to assume a deployed configuration, the resilient locking element is effective to engage another portion of the locking arrangement to retain the implant structure in the deployed configuration.

According to a further feature of the present invention, the implant structure includes two substantially opposite outward facing bone contact surfaces deployed for maintaining a predefined spacing between two adjacent bones of the human body, both of the bone contact surfaces being provided by the UHMWPE.

According to a further feature of the present invention, the implant structure is formed entirely from the UHMWPE.

According to a further feature of the present invention, the UHMWPE is cross-linked UHMWPE.

According to a further feature of the present invention, the implant structure has a generally arcuate form.

According to a further feature of the present invention, the implant structure forms a closed loop.

### BRIEF DESCRIPTION OF THE DRAWINGS

The invention is herein described, by way of example only, with reference to the accompanying drawings, wherein:

FIG. 1 is schematic isometric illustration of a spinal implant, constructed and operative according to the teachings of the present invention;

FIG. 2 is an isometric view of a first example of an interspinous process spacer, constructed and operative according to the teachings of the present invention, employing a structure corresponding to the X-STOP® spacer from Medtronics Inc. (US); and

FIGS. 3A and 3B are schematic isometric views of an additional example of an interspinous process spacer, constructed and operative according to the teachings of the present invention, show in a straightened and an arcuate deployed state respectively.

### DESCRIPTION OF THE PREFERRED EMBODIMENTS

The present invention is a spinal implant and corresponding method for implantation in a human spine in which a majority of the implant structure, including at least one bone contact surface, is formed from UHMWPE.

The principles and operation of implants according to the present invention may be better understood with reference to the drawings and the accompanying description.

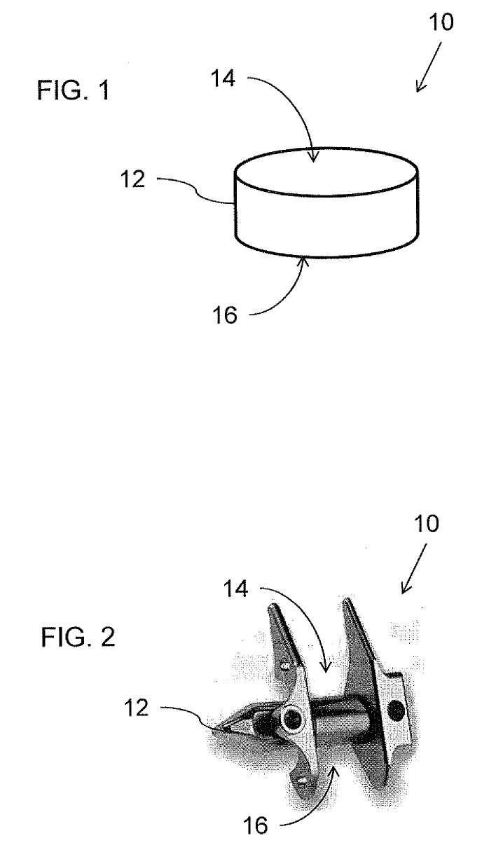

Referring now to the drawings, FIG. 1 shows a first implementation of a spinal implant, generally designated 10, constructed and operative according to the teachings of the present invention, for implantation in a human spine adjacent to at least one bone. Spinal implant 10 has an implant structure 12 including at least one bone contact surface 14, 16. It is a particularly preferred feature of the present invention that a majority of implant structure 12 by volume is formed from UHMWPE, and that UHMWPE provides the at least one bone contact surface 14, 16.

It will be noted that this structure departs markedly from the accepted thinking in the field of orthopedic implants. Specifically, as detailed above, UHMWPE is generally regarded as problematic for interfacing directly with bone at an exposed joint surface due to the well known problem of debris formation. Additionally, the use of UHMWPE as an external component without a surrounding support structure would be generally considered problematic due to the flow (creep) of the material that would be expected to occur, leading to variation of the dimensions of the device. According to the teachings of the present invention, it has been found that these factors are surprisingly not problematic in a range of spinal implant applications and, at least with regard to flow, may actually provide a number of advantages.

Specifically, without in any way limiting the scope of the present invention, it is believed that the problem of debris is largely avoided by deployment of the implant at locations in the spine where loading forces are exhibited primarily in the form of axial loading while sliding friction between the bone and the implant is typically much less than encountered in a conventional joint replacement employing UHMWPE as a sliding bearing surface. Furthermore, the opposing surface against which the UHMWPE rubs according to the teachings of the present invention is the bone itself, which is a much softer surface than the hard metal joint surfaces employed in conventional joint replacements. These factors are believed to combine to ensure that debris related complications are greatly reduced or even eliminated.

Regarding the issue of creep, it has been found that the proportional changes in dimensions encountered during normal usage in spinal applications are well within acceptable tolerances. In fact, any flow occurring in the UHMWPE implant actually improves performance of the implant, tending to conform at least in part to the local shape of the bone at the bone-implant interfaces and thereby spreading load more evenly and helping to avoid bone subsidence.

In addition, the properties of UHMWPE impart a number of other significant advantages to the implants of the present invention. Specifically, the implants exhibit excellent shock absorbing properties, mechanical robustness, flexibility, resilience, durability and tissue compatibility. Since the UHMWPE is more compressible than bone, it tends to absorb strain and protect the bone from damaging impacts.

The implant of FIG. 1 is illustrated schematically as a flattened disc. The actual geometry of the implant is varied according to the intended location for implantation, according to the required size, and according to the chosen fixation mechanism, all according to principles of operation which are generally known and currently practiced, and/or which are described herein. Thus, for example, the implant of FIG. 1 may be an intervertebral disc replacement. In this case, bone contact surfaces 14 and 16 are optionally modified by provision of suitable keels and/or other anchoring features to provide upper and lower endplates against which endplates of the inferior and superior vertebral bodies engage. In this case, the flexibility and resilience of the block of UHMWPE making up the implant structure provides mobility of the intervertebral joint, while at the same time, the UHMWPE contact surfaces provide good load distribution to minimize bone subsidence. Optionally, mobility can be further enhanced by forming the main block of UHMWPE with hollows or voids, for example generated during molding of the device or subsequent machining, thereby increasing the overall flexibility of the device.

Another field of application in which the spinal implants of the present invention may be used to advantage is the field of inter spinous process spacers. An example of such a spacer, commercially available under the trademark X-STOP® from Medtronics Inc. (US), is shown in FIG. 2. It is noted that the entire device is formed from hard metallic (titanium) components, typically leading to increased wear on the adjacent spinous processes, and risk of bone breakage, as the bone rubs against the metal during motion of the subject.

According to the teachings of the present invention, at least the tubular spacer element itself, and most preferably the entire structure, is formed from UHMWPE. As a result, both an inferior and a superior spinous process come in contact directly with the UHMWPE. Here too, the teaching of the present invention goes against the established thinking, given that direct contact of bone in a joint surface with UHMWPE would conventionally be expected to form an unacceptable source of debris generation.

Parenthetically, it will be noted that a particularly preferred but non-limiting subgroup of applications of the present invention relate to implants which are maintained in position by abutment with bone and other adjacent tissue, but without adhesion to any bone. This may be true of substantially all of the implants described herein.

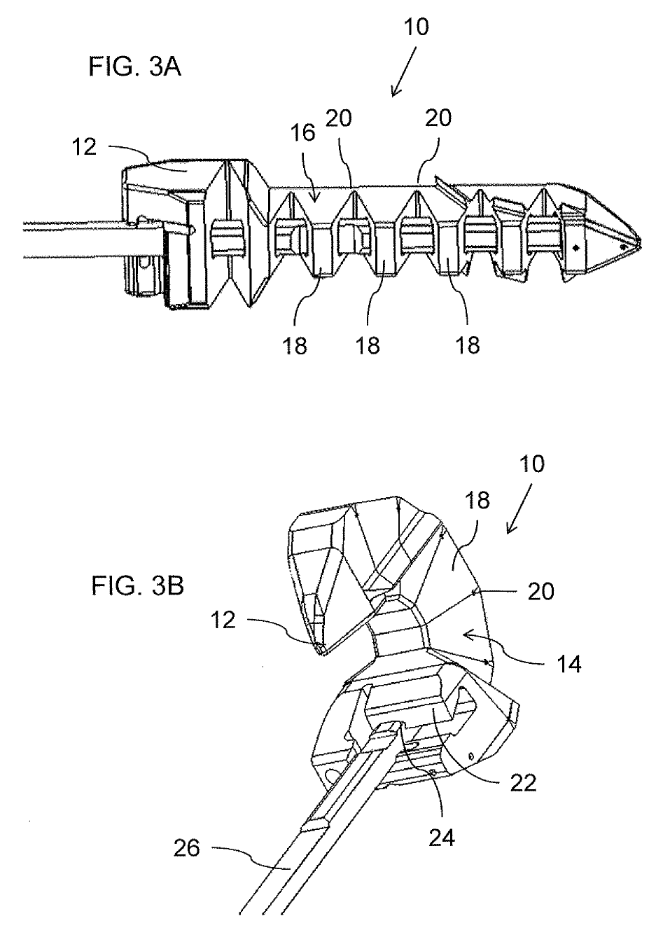

An alternative configuration of interspinous process spacer according to the present invention may be constructed according to the teachings of PCT Patent Application Publication No. WO 2009/019669 and as illustrated in FIGS. 3A and 3B. WO 2009/019669 is hereby incorporated by reference as if set out here in its entirety, but does not constitute prior art to the present invention. This implementation illustrates a feature of certain preferred implementations of the present invention according to which the implant structure includes a plurality of segments 18 interconnected by at least one integral hinge 20. Segments 18 and integral hinges 20 are integrally formed from UHMWPE, employing the inherent flexibility of the material to provide the hinge functionality. In the particular non-limiting case illustrated here, the implant structure is configured to assume a generally arcuate deployed form as shown.

Another feature of certain preferred implementations of the present invention is also exemplified by this embodiment, particularly as visible in FIG. 3B. Specifically, there is shown a locking arrangement including at least one resilient locking element, in this case, resilient tooth 22, resiliently biased to a locking position such that, when the implant structure is deflected by flexing at the at least one integral hinge to assume a deployed configuration (FIG. 3B), the resilient locking element is effective to engage another portion of the locking arrangement, in this case, a step 24 formed on a central tensioning element 26, to retain the implant structure in the deployed configuration. Thus, the locking mechanism employs the inherent resilient flexibility of UHMWPE to achieve effective locking of the device in its deployed configuration.

Although the present invention has been illustrated thus far with reference to various intervertebral disc replacements and interspinous process spacers, it should be noted that these implants are merely examples, and that the principles of the present invention may be applied to advantage in a wide range of other spinal implants. By way of additional non-limiting examples, implants according to the teachings of the present invention may be implemented according to the teachings of various embodiments of spinal implants described in PCT Patent Application Publication No. WO 2006/072941, which is hereby incorporated by reference as if set out here in its entirety. Specifically, reference is made to the various devices illustrated schematically in FIGS. 22A-29C thereof, all of which may be implemented according to the teachings of the present invention employing UHMWPE as the primary structural material. These structures also exemplify applications in which the implant structure is shaped for deployment at least partially within a vertebral body.

Other non-limiting exemplary applications of the present invention include: percutaneous interbody fusion (typically a hollow device filled with osteogenic potential); kyphoplasty (to treat osteoporotic fractures or to fill in pathological vertebral fractures); and treatments for scoliosis or kyphosis with asymmetric UHMWPE discs. It will be noted that the various applications of the present invention are relevant to all regions of the spinal column, whether cervical, thoracic or lumbar.

Similarly, the principles of the present invention may be applied to a wide range of other spinal implants, as will be clear to one ordinarily skilled in the art.

In all of the above implementations, the implant structure is most preferably formed entirely from UHMWPE. As mentioned earlier, the UHMWPE may optionally be cross-linked UHMWPE, thereby providing further enhanced resistance to wear and debris formation. One particularly preferred but non-limiting exemplary implementation employs UHMWPE which has undergone cross-linking with a 5 Mrad, +/−1 Mrad dosage of gamma radiation.

It will be appreciated that the above descriptions are intended only to serve as examples, and that many other embodiments are possible within the scope of the present invention as defined in the appended claims.

What is claimed is:

1. A spinal implant for implantation in a human spine adjacent to at least one bone, the spinal implant comprising an implant structure including at least one bone contact surface, wherein a majority of said implant structure by volume is formed from UHMWPE, and wherein said UHMWPE provides said at least one bone contact surface.

2. The spinal implant of claim 1, wherein said implant structure is shaped for deployment as an inter spinous process spacer such that both an inferior and a superior spinous process come in contact with said UHMWPE.

3. The spinal implant of claim 1, wherein said implant structure is shaped for deployment as an intervertebral disc replacement such that both an inferior and a superior vertebral body endplate come in contact with said UHMWPE.

4. The spinal implant of claim 1, wherein said implant structure is shaped for deployment at least partially within a vertebral body.

5. The spinal implant of claim 1, wherein said implant structure includes a plurality of segments interconnected by at least one integral hinge, said segments and said integral hinges being formed from said UHMWPE.

6. The spinal implant of claim 5, wherein said implant structure includes a locking arrangement including at least one resilient locking element resiliently biased to a locking position such that, when said implant structure is deflected by flexing at said at least one integral hinge to assume a deployed configuration, said resilient locking element is effective to engage another portion of said locking arrangement to retain said implant structure in said deployed configuration.

7. The spinal implant of claim 1, wherein said implant structure includes two substantially opposite outward facing bone contact surfaces deployed for maintaining a predefined spacing between two adjacent bones of the human body, both of said bone contact surfaces being provided by said UHMWPE.

8. The spinal implant of claim 1, wherein said implant structure is formed entirely from said UHMWPE.

9. The spinal implant of claim 1, wherein said UHMWPE is cross-linked UHMWPE.

10. The spinal implant of claim 1, wherein said implant structure has a generally arcuate form.

11. The spinal implant of claim 1, wherein said implant structure forms a closed loop.

12. A method for deploying a spinal implant comprising the steps of:
(a) providing an implant structure including at least one bone contact surface, wherein a majority of said implant structure by volume is formed from UHMWPE, and wherein said UHMWPE provides said at least one bone contact surface; and
(b) deploying said implant structure with said at least one bone contact surface in contact with a bone of a human spine.

13. The method of claim 12, wherein said implant structure is deployed as an inter spinous process spacer such that both an inferior and a superior spinous process come in contact with said UHMWPE.

14. The method of claim 12, wherein said implant structure is deployed as an intervertebral disc replacement such that both an inferior and a superior vertebral body endplate come in contact with said UHMWPE.

15. The method of claim 12, wherein said implant structure is deployed at least partially within a vertebral body.

16. The method of claim 12, wherein said implant structure includes a plurality of segments interconnected by at least one integral hinge, said segments and said integral hinges being formed from said UHMWPE.

17. The method of claim 16, wherein said implant structure includes a locking arrangement including at least one resilient locking element resiliently biased to a locking position such that, when said implant structure is deflected by flexing at said at least one integral hinge to assume a deployed configuration, said resilient locking element is effective to engage another portion of said locking arrangement to retain said implant structure in said deployed configuration.

18. The method of claim 12, wherein said implant structure includes two substantially opposite outward facing bone contact surfaces deployed for maintaining a predefined spacing between two adjacent bones, both of said bone contact surfaces being provided by said UHMWPE, and wherein said deploying includes deploying said implant structure between two adjacent bones of the human spine.

19. The method of claim 12, wherein said implant structure is formed entirely from said UHMWPE.

20. The method of claim 12, wherein said UHMWPE is cross-linked UHMWPE.

21. The method of claim 12, wherein said implant structure has a generally arcuate form.

22. The method of claim 12, wherein said implant structure forms a closed loop when deployed.
